(19) United States
(12) Patent Application Publication
(10) Pub. No.: US 2007/0123750 A1
(43) Pub. Date: May 31, 2007

(54) Catheter apparatus and methods of using same

(75) Inventors: Charles Edward Baumgartner, Niskayuna, NY (US); Mirsaid Mirsaid Seyed-Bolorforosh, Schenectady, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/290,020

(22) Filed: Nov. 30, 2005

(51) Int. Cl. A61B 1/00; A61B 5/05

(52) U.S. Cl. 600/143; 600/421

(57) ABSTRACT

A catheter apparatus includes a catheter having an electroactive polymer inside. The electroactive polymer is configured to control either or both shape and/or direction of the catheter in a vessel as the electroactive polymer is electrically activated. The apparatus further includes a computer which may be denoted as a “control computer.” The computer includes a memory and a processor configured to store vascular information and determine voltages to be applied to activate the electroactive polymer to steer the catheter to a desired site after it is inserted into a body. Also included is an electrical source responsive to the computer and configured to apply the determined voltages to the electroactive polymer.

### BACKGROUND OF THE INVENTION

This invention relates generally to medical devices, and more particularly to methods and apparatus for catheter devices that demand less skill for their manipulation inside a body.

Human vasculature possesses a highly branched structure. To place an ultrasound intracardiac probe into position within the heart, the imaging transducer is placed into a catheter and inserted into the vasculature, commonly through the leg or arm. Directing a catheter through the body's vascular network and into the heart necessitates having both the ability to visualize the vascular structure in real time and also the ability to steer the catheter tip in a desired direction. Careful steering of the cardiac tip is needed to avoid puncturing vascular walls.

A catheter contains a complex series of strings or wires attached to its tip at one end and to knobs on a handle at the other, opposite end. Careful turning of the knobs can pull the catheter tip in a desired direction, thereby allowing the catheter to be steered in that direction. A physician responsible for inserting the catheter must simultaneously insert and steer the catheter while monitoring the location of the catheter tip fluoroscopically.

An ultrasound intracardiac catheter probe must be directed through vasculature and into a heart to be placed in the appropriate location for imaging. The placement is accomplished by steering the catheter tip as the catheter is inserted within the vascular system. This steering is generally done using a series of wires or cords attached to the catheter tip and controlled manually from the catheter handle as the catheter is inserted into (for example) an artery or vein in a leg of a patient. The location of the catheter during insertion is followed fluoroscopically. A physician requires much skill to move the catheter tip into position without damaging the vasculature or heart walls.

### BRIEF DESCRIPTION OF THE INVENTION

In one aspect, the present invention therefore provides a catheter apparatus. The catheter apparatus includes a catheter having an electroactive polymer inside. The electroactive polymer is configured to control either or both shape and/or direction of the catheter in a vessel as the electroactive polymer is electrically activated. The apparatus further includes a computer which may be denoted as a “control computer.” The computer includes a memory and a processor configured to store vascular information and determine voltages to be applied to activate the electroactive polymer to steer the catheter to a desired site after it is inserted into a body. Also included is an electrical source responsive to the computer and configured to apply the determined voltages to the electroactive polymer.

In another aspect, the present invention provides a method for imaging an organ of a body. The method includes imaging a vascular system of the body, digitizing and storing vascular information obtained from the imaging in a control computer, and using the control computer to apply voltages to an electroactive polymer in a catheter in accordance with the stored vascular information to steer the catheter to a desired site after it is inserted into the body. (Fluoroscopy and digital fluoroscopy are examples of vascular imaging systems.)

In yet another aspect, the present invention provides a catheter apparatus that includes a catheter having an electroactive polymer inside. The electroactive polymer is configured to control either shape and/or direction of the catheter in a vessel as the electroactive polymer is electrically activated. The apparatus further includes a contact sensor configured to sense contact with a vessel wall or an organ wall. Also included is a control computer responsive to the contact sensor and configured to utilize an electrical source to immediately electrically activate the electroactive polymer to change shape or direction of the catheter upon the contact sensor sensing contact with a vessel wall or an organ wall.

It will be appreciated that various configurations of the present invention provide methods and apparatus having or using catheters with very small, if any, width beyond those of prior art catheters. Catheters are easily controlled and can be automatically controlled by computer in some configurations.

### BRIEF DESCRIPTION OF THE DRAWINGS

FIG. 1 is a schematic pictorial view of a catheter apparatus configuration of the present invention, with a portion of the catheter itself cut away for viewing of internal electroactive polymer bundles.

FIG. 2 is a schematic pictorial view of the catheter of FIG. 1 being manipulated in a body.

FIG. 3 is a partial cut away view of the catheter of FIG. 1 showing the internal electroactive polymer bundles in somewhat greater clarity.

FIG. 4 is a flow chart of a method configuration of the present invention.

### DETAILED DESCRIPTION OF THE INVENTION

In some configurations of the present invention, a catheter tip is steered using a plurality of electroactive polymer bundles along a length of the catheter. Electroactive polymers change their size in response to an electrical voltage and are sometimes referred to as “artificial muscles.” A series of electroactive polymers formed as cords and attached to electrical leads along the catheter can be made to independently stretch or shrink in response to an applied voltages, resulting in bending or steering of the catheter in a desired direction. For example, in some configurations of the present invention, the vascular system is imaged using a contrast agent fluoroscopic method. The vascular information obtained is digitized and stored in a control computer. The computer then applies voltages to a catheter tip in accordance with the stored vascular information to steer the catheter to a desired site after it is inserted into the body. Thus, the skill and dexterity required to steer the catheter is substantially reduced, as the steering is entirely, or at partially, performed under automatic electronic control.

Also in some configurations, a series of cords that are formed from or that comprise electroactive polymers plus appropriate electrical connectors is placed within an intravascular catheter. A voltage is applied to individual electrical connections to individual cords using, for example, a touch panel or any other suitable electronic control and/or pointing device. As voltages are varied, the cords change shape accordingly. By varying the voltages applied to individual cords, the shape of the cords change shape, thereby steering a tip of the catheter in a desired direction.

Some configurations of the present invention have more than one set of electroactive polymer sets and controls along the length of the catheter. In these configurations, the shape of the catheter can be varied more than in those configurations in which guide wires are attached to the catheter tip.

Also, in some configurations, the shape of a vascular network through which the catheter is to be inserted is accurately ascertained using digitized x-ray fluoroscopic methods and contrast agents. Coordinates representing the shape of the vascular network are digitized and used by a “control computer” to generate electrical signals that control the location of the catheter during insertion. Thus, much of the skill required in known catheterization techniques can be supplied by the computer rather than the physician. Also, some configurations include a location sensor at the tip of the catheter to aid a computer in steering the catheter tip.

A contact sensor is provided in some configurations of the present invention for sensing contact with walls of the heart or vasculature. Upon contact, the contact sensor generates an electrical signal that is detected by the control computer. Once contact with the vasculature is detected, the computer or other control device causes a voltage change on an electroactive polymer cord that results in the catheter tip immediately changing shape slightly to avoid or prevent damage to cardiac or vessel walls.

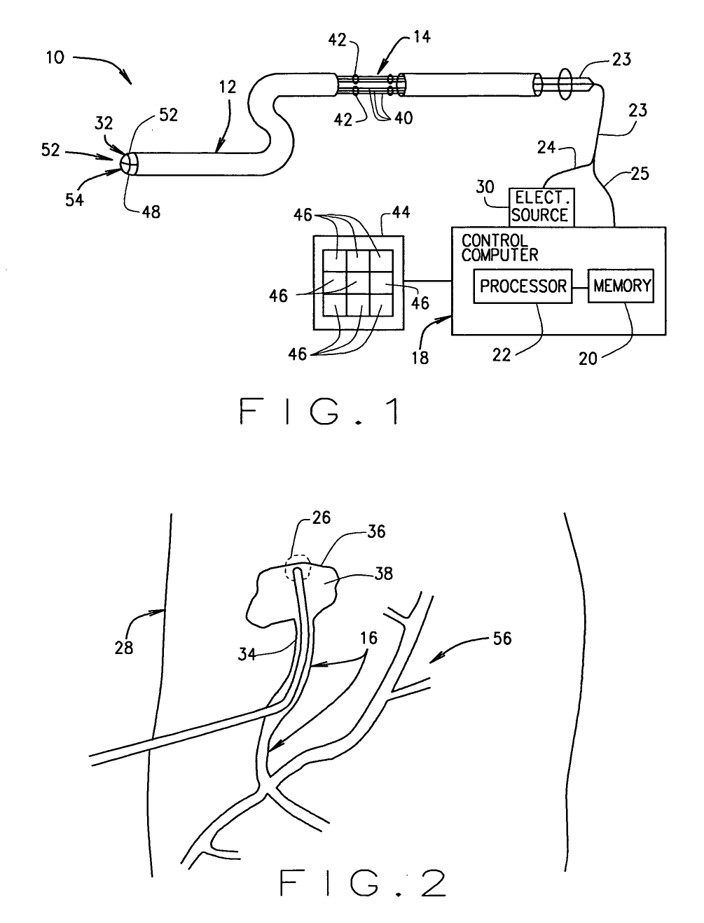

In some configurations and referring to FIGS. 1 and 2, a catheter apparatus 10 comprising a catheter 12 has an electroactive polymer 14 therein. Electroactive polymer 14 is configured to control the shape and/or direction of catheter 12 in a vessel 16 as electroactive polymer 14 is electrically activated. (The term “and/or” is intended to encompass configurations in which only one of shape or direction is controlled, as well as configurations in which both can be controlled.) A computer 18 (which need not be a general purpose computer or PC in all configurations) including a memory 20 and a processor 22 configured to store vascular information and determine voltages to be applied to activate electroactive polymer 14 to steer catheter 12 to a desired site 26 after it is inserted into a body 28. An electrical source 30 is responsive to computer 18 and is configured to apply the determined voltages to electroactive polymer 14. An electric cable 23 from catheter 12 includes wires 24 carrying one or more electrical voltages from electrical source 30 to electroactive polymer 14, and wires 25 carrying one or more sensing signals output by catheter 12.

Also in some configurations, catheter 12 further comprises a contact sensor 32 configured to sense contact with a wall 34 of a vessel 16 wall or wall 36 of an organ 38. Computer 18 is responsive to contact sensor 32 and is configured to utilize electrical source 30 to immediately electrically activate electroactive polymer 14 to change the shape and/or direction of catheter 12 upon contact sensor 32 sensing contact with a vessel wall 34 or an organ wall 36.

Referring to FIG. 2, electroactive polymer 14 in some configurations comprises a plurality of cords 40, wherein each cord 40 is electrically controllable to adjust the shape and/or direction of catheter 12. In some configurations, the plurality of cords 40 are arranged in bundles 42.

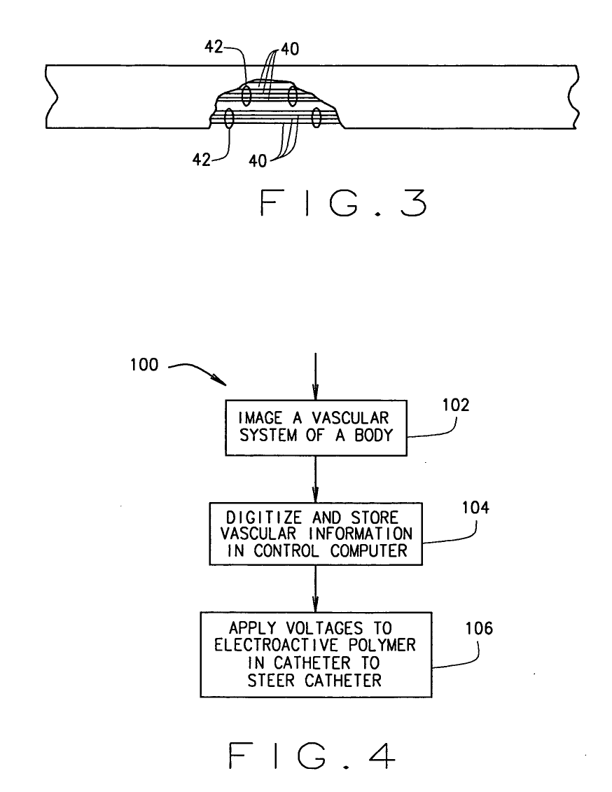

In some configurations and referring to FIG. 3, apparatus 10 also includes a touch panel 44 (i.e., a panel comprising push buttons, a keyboard, capacitive sensors, pressure-sensitive areas, or other types of switches 46 that are touch and/or pressure sensitive) configured to control a voltage applied to each cord 40. For example, computer 18 is responsive to switches 46 on touch panel 44 to control a voltage supplied by electrical source 30 to each cord 40, so that manual control of the direction and/or shape of catheter 12 can be accomplished easily.

Some configurations of apparatus 10 have a plurality of separately controllable cords 40 along all or a portion of the length of catheter 12.

In many configurations, a contrast enhanced tip (“location sensor”) 48 is provided at tip 50 of catheter 12. This tip is imagable with fluorscopy during catheter insertion into the vasculature and aids the control computer in determining correct catheter placement.

To provide images inside body 28, catheter 12, in some configurations, includes an ultrasound imaging transducer 52 and/or an optical imaging transducer 54.

In some configurations and referring to flow chart 100 of FIG. 3, a configuration of a method to image an organ of a body includes imaging 102 a vascular system 56 of body 28. The vascular information obtained from this imaging is digitized and stored 104 in a control computer 18. Control computer 18 is then utilized to apply 106 voltages to electroactive polymer 14 in a catheter 12 in accordance with the stored vascular information to steer catheter 12 to a desired site 26 after it is inserted into body 28.

In some configurations, to image 102 a vascular system 56 of body 28 , a contrast agent is used to fluoroscopically image vascular system 56.

Electroactive polymer 14 is, in some configurations, configured to change at least one of shape or direction of catheter 12 when electrically activated, and catheter 12 further comprises a contact sensor 32 configured to sense contact with a vessel wall 34 or an organ wall 36. For example, contact sensor 32 generates a signal that is sent to control computer 18 whenever contact is made. Control computer 18 is used to immediately activate electroactive polymer 14 to change shape or direction of catheter 12 upon contact sensor 32 sensing contact with a vessel wall 34 or an organ wall 36. More than one contact sensor 32 can be used to determine the orientation of tip 50 with respect to vessel wall 34 or organ wall 36.

In some configurations, catheter 12 has a plurality of cords 40 therein that are comprised of electroactive polymer 14, and the method further includes electrically controlling each cord 40 to adjust the shape and/or direction of catheter 12.

In some configurations, a touch panel 44 is utilized to control a voltage applied to each cord 40 to manually steer catheter 12.

Catheter 12 has a plurality of separately controllable cords 40 along its length in some configurations, so that some method configurations further comprise separately controlling each controllable cord 40.

An ultrasound imaging transducer 54 or an optical imaging transducer 56 located at a tip 50 of catheter 12 is used in some method configurations of the present invention to produce an image.

In some configurations of the present invention and again referring to FIGS. 1 and 2, a catheter apparatus 10 is provided that includes a catheter 12 having an electroactive polymer 14 therein. Electroactive polymer 14 is configured to control at least one of shape or direction of catheter 12 in a vessel 16 as electroactive polymer 14 is electrically activated. The apparatus also includes a contact sensor 32 figured to sense contact with a vessel wall 34 an organ wall 36 and a control computer 18 responsive to contact sensor 32. Control computer 18 is configured to utilize an electrical source 30 to immediately electrically activate electroactive polymer 14 to change shape or direction of catheter 12 upon contact sensor 32 sensing contact with a vessel wall 34 or an organ wall 36. Additionally, some of these configurations also include at least one of an ultrasound imaging transducer 52 or an optical imaging transducer 56.

It thus be appreciated that various configurations of the present invention can be provide with very small, if any, width beyond those of prior art catheters. Moreover, configurations of the present invention are easily controlled and can be automatically controlled by computer in some configurations.

While the invention has been described in terms of various specific embodiments, those skilled in the art will recognize that the invention can be practiced with modification within the spirit and scope of the claims.

What is claimed is:

1. A catheter apparatus comprising a catheter having an electroactive polymer therein, the electroactive polymer configured to control at least one of shape or direction of the catheter in a vessel as the electroactive polymer is electrically activated;
a computer including a memory and a processor configured to store vascular information and determine voltages to be applied to activate the electroactive polymer to steer the catheter to a desired site after it is inserted into a body; and
an electrical source responsive to the computer and configured to apply the determined voltages to the electroactive polymer.

2. An apparatus in accordance with claim 1 wherein the catheter further comprises a contact sensor configured to sense contact with a vessel wall or an organ wall, and the computer responsive to the contact sensor and configured to utilize the electrical source to immediately electrically activate the electroactive polymer to change shape or direction of the catheter upon the contact sensor sensing contact with a vessel wall or an organ wall.

3. An apparatus in accordance with claim 1 having a plurality of cords therein comprised of the electroactive polymer, wherein each said cord is electrically controllable to adjust at least one of the shape or direction of the catheter.

4. An apparatus in accordance with claim 3 wherein the plurality of cords are arranged in bundles of cords.

5. An apparatus in accordance with claim 3 further comprising a touch panel configured to control a voltage applied to each said cord.

6. An apparatus in accordance with claim 3 having a plurality of separately controllable cords along a length of the catheter.

7. An apparatus in accordance with claim 1 having a tip, and further comprising a location sensor at the tip.

8. An apparatus in accordance with claim 1 further comprising an ultrasound imaging transducer.

9. An apparatus in accordance with claim 1 further comprising an optical imaging transducer.

10. A method for imaging an organ of a body, the method comprising:
imaging a vascular system of the body;
digitizing and storing vascular information obtained from said imaging in a control computer; and
utilizing the control computer to apply voltages to electroactive polymer in a catheter in accordance with the stored vascular information to steer the catheter to a desired site after it is inserted into the body.

11. A method in accordance with claim 10 wherein said imaging a vascular system of the body comprises utilizing a contrast agent to fluoroscopically image the vascular system.

12. A method in accordance with claim 10 wherein the electroactive polymer is configured to change at least one of shape or direction of the catheter when electrically activated, and the catheter further comprises a contact sensor configured to sense contact with a vessel wall or an organ wall,
and further comprising utilizing the control computer to immediately activate the electroactive polymer to change shape or direction of the catheter upon the contact sensor sensing contact with a vessel wall or an organ wall.

13. A method in accordance with claim 10 wherein the catheter has a plurality of cords therein comprised of electroactive polymer, the method further comprising electrically controlling each said cord to adjust at least one of the shape or direction of the catheter.

14. A method in accordance with claim 13 further comprising utilizing a touch panel to control a voltage applied to each cord.

15. A method in accordance with claim 13 wherein the catheter has a plurality of separately controllable cords along its length, and the method further comprises separately controlling each said controllable cord.

16. A method in accordance with claim 10 further comprising using the processor to immediately change the shape of the catheter upon the contact sensor sensing contact with a vessel wall or an organ wall.

17. A method in accordance with claim 10 further comprising utilizing an ultrasound imaging transducer located at a tip of the catheter to produce an image.

18. A method in accordance with claim 10 further comprising utilizing an optical imaging transducer located at a tip of the catheter to produce an image.

19. A catheter apparatus comprising:
a catheter having an electroactive polymer therein, the electroactive polymer configured to control at least one of shape or direction of the catheter in a vessel as the electroactive polymer is electrically activated;
a contact sensor configured to sense contact with a vessel wall or an organ wall;
and a control computer responsive to the contact sensor and configured to utilize an electrical source to immediately electrically activate the electroactive polymer to change shape or direction of the catheter upon the contact sensor sensing contact with a vessel wall or an organ wall.

20. An apparatus in accordance with claim 1 further comprising at least one of an ultrasound imaging transducer or an optical imaging transducer.
